(19) United States
(12) Patent Application Publication
(10) Pub. No.: US 2022/0260189 A1
(43) Pub. Date: Aug. 18, 2022

(54) STERILE CONNECTOR FOR THE STERILE TRANSFER OF A LIQUID MEDIUM

(71) Applicant: Sartorius Stedim Biotech GmbH, Göttingen (DE)

(72) Inventor: Mario DEUSE, Bovenden (DE)

(73) Assignee: Sartorius Stedim Biotech GmbH, Göttingen (DE)

(21) Appl. No.: 17/621,973

(22) PCT Filed: Jun. 5, 2020
(86) PCT No.: PCT/EP2020/065686

§ 371 Date: Dec. 22, 2021

(30) Foreign Application Priority Data
Jun. 24, 2019 (DE) 10 2019 116 970.4

(51) Int. Cl. F16L 29/00; B01L 3/00; F16L 33/02

(52) U.S. Cl. CPC F16L 29/005; B01L 3/563; F16L 2201/44; B01L 2200/141; F16L 33/02

(57) ABSTRACT

The invention relates to a sterile connector for the sterile transfer of a liquid medium, in particular a biological medium, from a liquid container (2) into a fluid chamber (3, 3), wherein the sterile connector (1) has a first coupling device (5) and a second coupling device (6). It is proposed that the first coupling device (5) has a fluid inlet (7) and a fluid outlet (8) and also a first cannula (9), the latter having an end which is directed away from the fluid inlet (7) and forms the fluid outlet (8), that the second coupling device (6) has a fluid passage (10) which, in an initial state of the sterile connector (1), is covered axially by a first septum (11), that the fluid outlet (8), in the initial state of the sterile connector (1), is arranged in a hermetically sealed region of the first coupling device (5) and the surface (11a) of the first septum (11) facing axially away from the fluid passage (10) is arranged in a hermetically sealed region of the second coupling device (6), and that, in a fluidic connection process starting from the initial state of the sterile connector (1), a fluidic connection between the fluid inlet (7) and the fluid passage (10) can be produced by the fact that the end of the first cannula (9) forming the fluid outlet (8) pierces the first septum (11) of the second coupling device (6).

The invention concerns a sterile connector for the sterile transfer of a liquid, in particular biological medium, according to the preamble of claim 1, a packing arrangement with a packing and at least one first or second coupling device of such a sterile connector packed in sterile fashion therein, or such a sterile connector packed in sterile fashion therein according to claim 26, and the use of such a sterile connector packed in sterile fashion, a first or second such coupling device packed in sterile fashion, and/or such a packing arrangement according to claim 28.

An application example for the sterile connector is the sterile transfer of a liquid, in particular biological medium, into a bioprocess-technical system. A bioprocess-technical system here generally means a device with which biotechnological processes can be carried out or supported. Purely as an example, bioreactors are cited here, in which microorganisms or tissue cells are cultivated under predefined conditions. Such a device regularly comprises a container containing a biological reaction medium which is composed of the substances provided for a biotechnological process, e.g. microorganisms or tissue cells on one side and a corresponding nutrient medium on the other, in order to be able to perform the respective biotechnological process step, for example fermentation or cultivation.

An example of such a bioreactor is firstly a production bioreactor, i.e. a relatively large bioreactor on production scale with a working volume of for example several hundred or several thousand liters, for industrial production of microbial or cellular products, in particular biopharmaceuticals. Such a bioreactor forms as a product a fermentation broth which is normally processed further in a so-called downstream process in order to obtain a product from the cells or the culture supernatant. Another example is a laboratory bioreactor, i.e. a relatively small bioreactor on laboratory scale with a working volume of for example less than 10 liters. Such a laboratory bioreactor serves for example for production of ATMPs (Advanced Therapy Medical Products) and/or for performance of cell expansion, by means of which a sufficient number of cells for the respective application, in particular tissue cells or microbial cells, can be produced. One application for this is the multiplication of human cells, for example T-cells (T-lymphocytes) which are taken from the patient then expanded ex vivo and re-infused into the patient.

In particular in the manufacture of ATMPs with a laboratory bioreactor, but in principle also in a production bioreactor, particular importance is laid on a sterile addition of the individual liquid, e.g. biological, media to the respective bioreactor. Since many ATMPs cannot be filtered in sterile fashion or later sterilized, such medicaments are manufactured in sterile fashion in clean rooms of a corresponding clean room class. Thus the addition of a liquid medium to a bioreactor or the transfer of a liquid medium between two culture vessels and/or bioreactors in principle carries a risk of contamination, for which reason such handling steps are usually carried out in a clean room of clean room class A (GMP guideline Annex 1) or ISO 5 (ISO 14644-1). The production of medicaments in such clean rooms is however very cost-intensive because of the high monitoring requirements, complex clothing procedures etc.

For this reason, there is a demand for closed systems for cultivation or expansion of cells, in particular in the field of manufacture of ATMPs, whereby clean rooms of a lower clean room class may be used, for example clean rooms of clean room class D (GMP guideline Annex 1) or ISO 8 (ISO 14644-1). One challenge however is the addition of liquid medium to such a closed system as simply as possible. Various connecting systems are known such as Luer connectors, Luer lock connectors etc., in order for example to be able to couple a small capacity liquid container, such as a syringe or vial, to a closed system. At the coupling point at which the two coupling devices of the connector are connected together—for a Luer connector, the male Luer terminal and the female Luer terminal—there is a risk of contamination, for which reason particular attention must be paid to sterile conditions in the creation of the connection.

The invention is based on the problem of designing or refining a sterile connector for the sterile transfer of a liquid medium from a liquid container to a fluid chamber, for example of a bioprocess-technical system, such that sterile conditions can easily be created on transfer of the medium.

The above problem is solved with a sterile connector for the sterile transfer of a liquid, in particular biological medium according to the preamble of claim 1, with the features of the characteristic part of claim 1.

An essential feature is the fundamental consideration of creating a sterile connector with two coupling devices to be coupled together for the liquid transfer from a liquid container, which is for example a minimal quantity liquid container, to a fluid chamber, in which the coupling devices are initially provided in a state in which contamination of the parts decisive for the liquid transfer by the user is simply not possible, since in this state they are concealed within the coupling device and thereby protected from contact. The fluid chamber in which the liquid medium is filled from the liquid container via the sterile connector may be formed by a bioprocess-technical system, in particular a bioreactor. In principle however, it is also possible that the fluid chamber forms part of the second coupling device, i.e. the coupling device facing away from the liquid container. In the latter case, the fluid chamber is preferably closed apart from a fluid passage via which the liquid medium is conducted into the fluid chamber. The fluid chamber may however also open into a further fluid outlet which is or can be fluidically connected to a bioprocess-technical system, in particular a bioreactor.

With the sterile connector as proposed, a first preferably sterile cannula of a first coupling device assigned to the liquid container, in any case with its front end forming a fluid outlet, is initially in a position protected from contamination. In a further second coupling device, a first septum is arranged which axially covers and/or closes the fluid passage in this coupling device and thereby also protects this from contamination. The first septum is then pierced by the first cannula in an activation process which creates a fluidic connection through the entire sterile connector, whereby then the liquid medium can pass via the first cannula through the fluid passage. In order to be able to come into contact with the first septum, firstly access must be created through a second septum, which takes place automatically by a coupling process in which the two coupling devices are correctly connected together. During this coupling process, a second, preferably sterile cannula, in or into which the first cannula is movable, pierces said second septum. Until piercing of the second septum, the second cannula may still be arranged in a sealing envelope which is also pierced thereby on piercing of the second septum. In an activation process following the coupling process, now the first cannula may be guided through the second cannula up to the first septum and then pierce this. Until piercing of the first septum, the first cannula may still be arranged in a sealing envelope which is also pierced thereby during piercing of the first septum. The proposed cannulas are preferably made of metal for optimal piercing of the respective septum, but in principle may also be made of a plastic material. Particularly preferably, at their end facing the respective septum, the cannulas are pointed, but in principle may also be blunt.

Contamination of the first cannula in the region of the fluid outlet, i.e. in the region of its front end, is thus not possible until the time at which the first cannula comes into contact with the first septum. The first septum also cannot be contaminated until this time, since in the initial state of the sterile connector, i.e. before performance of the coupling process, it is arranged in a preferably sterile region protected from contamination. Also, the fluid passage in the second coupling device is sterile and protected from contamination until the time at which the first cannula punctures the first septum. This ensures that no germs or other media can reach the liquid medium during the transfer.

It is particularly preferred that before interconnection of the two coupling devices, i.e. before the coupling process, the liquid container from which medium is to be extracted, and/or a bioprocess-technical system to which the medium is to be added, are connected in sterile fashion to the respective assigned coupling device. Particularly preferably, this takes place by the manufacturer. Thus it is conceivable that the manufacturer of the liquid container already provides this with the assigned first coupling device of the sterile connector, wherein the liquid container particularly preferably is already filled by the manufacturer at this time, wherein the liquid container and the first coupling device are preferably made available to the user in a correctly interconnected state, in particular as a single-use component. The same also applies to the bioprocess-technical system, wherein it is also conceivable that the manufacturer of the system has already provided this with the assigned second coupling device of the sterile connector, in some cases with several such coupling devices, wherein the bioprocess-technical system and the second coupling device are preferably made available to the user in the correctly interconnected state, in particular a single-use components.

In this way, the two coupling devices of the sterile connector cannot be contaminated at any point decisive for the liquid transfer before creation of the connection, since there is already a sterile connection in the region in which the medium passes from the liquid container to the first coupling device connected thereto, and in the region in which the medium passes from the further second coupling device into the bioprocess-technical system. The portions or elements of the coupling devices, via which the liquid medium passes correctly from the coupling device assigned to the liquid container into the respective other coupling device, are also protected from contamination until the time of liquid transfer. In this way, via the sterile connector as proposed, in a particularly simple fashion, a sterile connection may be created between a liquid container and a fluid chamber, in particular a bioprocess-technical system.

It should be pointed out again that the liquid chamber need not necessarily be part of a bioprocess-technical system, but may also be part of the second coupling device and in particular may be closed with the exception of the fluid passage through which the liquid medium enters the fluid chamber.

In detail, it is now proposed that the first coupling device has a fluid inlet and a fluid outlet fluidically connected thereto, wherein the first coupling device comprises a first, preferably sterile cannula having an end which faces away from the fluid inlet and forms the fluid outlet; the second coupling device has a fluid passage which, in an initial state of the sterile connector, is axially covered by a first septum; in the initial state of the sterile connector, the fluid outlet formed by the first cannula is arranged in an in particular sterile region of the first coupling device which is hermetically sealed against the environment of the first coupling device, and the surface of the first septum axially facing away from the fluid passage is arranged in an in particular sterile region of the second coupling device which is hermetically sealed against the environment of the second coupling device; and in a fluidic connection process, starting from the initial state of the sterile connector, a fluidic connection can be created between the fluid inlet of the first coupling device and the fluid passage of the second coupling device, in that the end of the first cannula forming the fluid outlet in any case pierces the first septum of the second coupling device.

The terms “hermetically tight” or “hermetically sealed” in this context mean that a seal is provided which prevents a penetration of contaminants, in particular germs. In a further embodiment, the terms “hermetically tight” or “hermetically sealed” refer to a sterile and/or contamination-protected region of the sterile connector. In a particularly preferred embodiment, the terms “hermetically tight” or “hermetically sealed” also comprise the term “sterile” and are therefore interchangeable.

Claims 2 and 3 define in more detail the fluidic connection process in which a fluidic connection is created between the fluid inlet of the first coupling device and the fluid passage of the second coupling device. Thus the fluidic connection process is firstly a coupling process in which the first coupling device is mechanically connected to the second coupling device (claim 2), and secondly an activation process in which the first coupling device is adjusted relative to the second coupling device from a starting position into an operating position (claim 3). The adjustment comprises preferably a relative linear movement and in some cases also a relative rotational movement of the two coupling devices relative to one another. The coupling process here serves in particular exclusively to create a mechanical connection, whereas the activation process serves in particular exclusively to create a fluidic connection.

Claims 4 to 10 concern the coupling process and describe preferred embodiments and action mechanisms of the elements of the sterile connector which cooperate during the coupling process. Particularly preferably, here the region of the second coupling device which in the initial state is hermetically sealed, is delimited at least partially, preferably completely, by the first septum and a second septum (claim 4). Via a second, preferably sterile cannula of the first coupling device (claim 5), then the coupling process creates access for the first cannula to the hermetically sealed region of the second coupling device. Particularly preferably here, in the initial state, the second cannula is surrounded by a first sealing envelope, in particular in the form of a bellows, which the second cannula pierces during the coupling process (claim 8), in particular directly before piercing the second septum. The movement which causes the piercing of the second septum and in particular of the first sealing envelope by the second cannula may be transmitted to the second cannula by a cannula holder, which is mounted so as to be axially movable in the first coupling device (claim 9).

Claims 11 to 14 concern the activation process and describe preferred embodiments and action mechanisms of the elements of the sterile connector which cooperate during the activation process. The first coupling device here preferably has a second sealing envelope, which in any case surrounds the portion of the first cannula which during the activation process pierces the first septum (claim 11). The second sealing envelope is here preferably arranged together with the first cannula inside the second cannula, wherein during the coupling process, the second cannula can be moved axially relative to the first cannula and the second sealing envelope, so that the front end of the first cannula and the second sealing envelope protrude axially from the second cannula, however preferably only after the second cannula has pierced the second septum. The activation process may cause an axial cannula portion of the first cannula, which in particular surrounds the front cannula end or fluid outlet, to pierce the second sealing envelope (claim 12), wherein the first cannula then, preferably immediately afterwards, pierces the first septum. To perform the activation process, at least one actuation element may be provided on the cannula holder (claim 13), by means of which the cannula holder and accordingly the second cannula, together with the second coupling device mechanically connected thereto, can be moved linearly and in particular also rotationally.

Claim 15 concerns a fluid chamber of the second coupling device into which the fluid passage of the second coupling device opens. This fluid chamber may be fluidically connected to a fluid outlet of the second coupling device or otherwise closed. The term “otherwise closed” means that the fluid chamber has no further opening other than the fluid passage and to this extent forms a further liquid container. Liquid media may also be stored and/or mixed in such a liquid container, as in a bioprocess-technical system. Preferably, the fluid chamber of the second coupling device is however merely a component through which the liquid medium is conducted as it flows via the first coupling device from the liquid container connected thereto through the fluid passage of the second coupling device, in particular to a bioprocess-technical system, for example a bioreactor.

Claims 16 to 25 concern the fluidic connection process of a further embodiment of the sterile connector, and describe preferred embodiments and action mechanisms of the elements of the sterile connector which cooperate during the coupling process and the activation process. The adjustment during the activation process here again comprises a relative linear movement, but in contrast to the embodiment described above, preferably has no relative rotational movement of the two coupling devices relative to one another (claim 16).

Particularly preferably, in this further embodiment of a sterile connector, the region of the second coupling device which is hermetically sealed in the initial state is delimited by the first septum and by a first sealing envelope, in particular in the form of a bellows (claim 17). Via a second, preferably sterile cannula of the second coupling device (claim 18), the coupling process may then provide access for the first cannula to the first septum (claim 20). Particularly preferably, an axial cannula portion of the second cannula which pierces the second septum during the coupling process, is surrounded in the initial state by the first sealing envelope (claim 21), and in particular immediately before piercing of the second septum.

According to a further embodiment in claim 25, in the further embodiment a mechanism is provided for indication, in particular haptic indication, of at least one defined axial position of the first coupling device relative to the second coupling device. Preferably, in the respective defined axial position, an axial form fit is created, in particular via a catch mechanism, between the first coupling device and the second coupling device. A defined axial position in particular corresponds to the starting position in coupled state, and/or a defined axial position corresponds to the operating position in coupled state, i.e. the state after completion of the activation process.

According to a further teaching in claim 26, which has independent significance, a packing arrangement is claimed with a packing and with, packed in sterile fashion therein, at least one, preferably precisely one, first or second coupling device of a sterile connector as proposed, or with, packed in sterile fashion therein, a sterile connector as proposed. In view of the fact that the packing arrangement as proposed comprises a sterile connector as proposed or a coupling device of a sterile connector as proposed, reference may be made to all statements of the aforementioned teaching in this respect.

According to claim 27, the packing may also contain, packed in sterile fashion, either a liquid container or a bioprocess-technical system, wherein the liquid container or the bioprocess-technical system is already fluidically connected to the respective coupling device in the packing.

According to a further teaching in claim 28, which also has independent significance, the use is claimed of a sterile connector as proposed, packed in sterile fashion, a coupling device, packed in sterile fashion, of a sterile connector as proposed, and/or a packing arrangement as proposed, for the sterile transfer of liquid, in particular biological medium from a liquid container into a fluid chamber, preferably of a bioprocess-technical system, in particular a bioreactor, or into a fluid chamber of the second coupling device. In view of the fact that the use as proposed concerns the use of a sterile connector as proposed, a coupling device of a sterile connector as proposed, and/or a packing arrangement as proposed, reference may be made to all statements of the aforementioned teaching in this respect.

With the packing arrangement as proposed and the use as proposed, it has been found that, with the sterile connector as proposed or with the coupling devices of the sterile connector as proposed, which according to the proposal are also claimed as such, a liquid medium can be supplied particularly simply without significant risk of contamination to a closed system, in particular a bioreactor, in that the coupling devices keep the parts decisive for the liquid transfer, in particular said first cannula and the fluid passage, in a sterile state, namely in the coupled state in said starting position, until the liquid transfer actually takes place, namely in the operating state established by the activation process.

In the particularly preferred embodiment according to claim 29, individual ones of said components are preferably configured as single-use components, wherein the components are preferably made of a plastic material.

The invention is explained in more detail below with reference to a drawing showing merely one exemplary embodiment. The drawing shows:

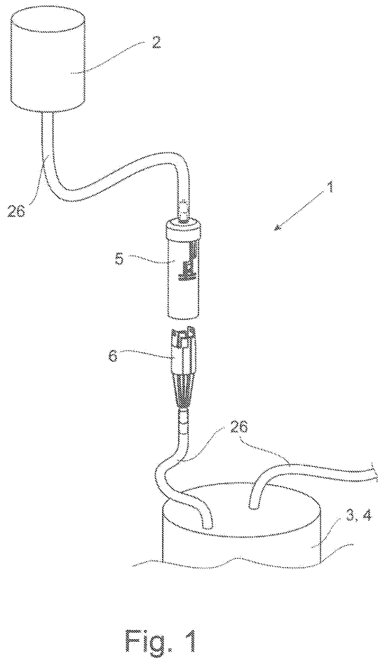

FIG. 1 in a perspective view, a sterile connector as proposed according to a first exemplary embodiment during connection of its two coupling devices,

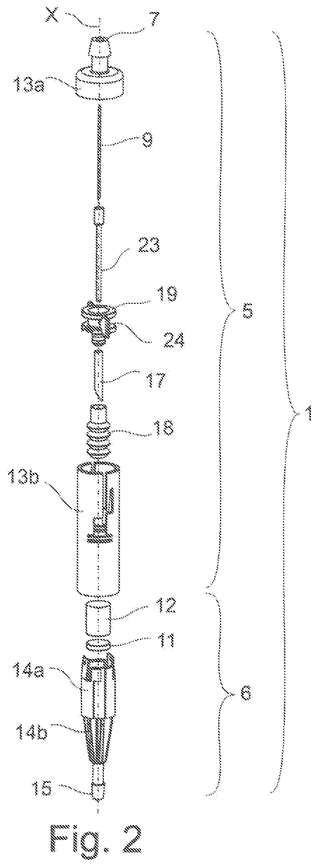

FIG. 2 in a perspective view, the sterile connector as proposed according to FIG. 1 with its two coupling devices, in an exploded illustration,

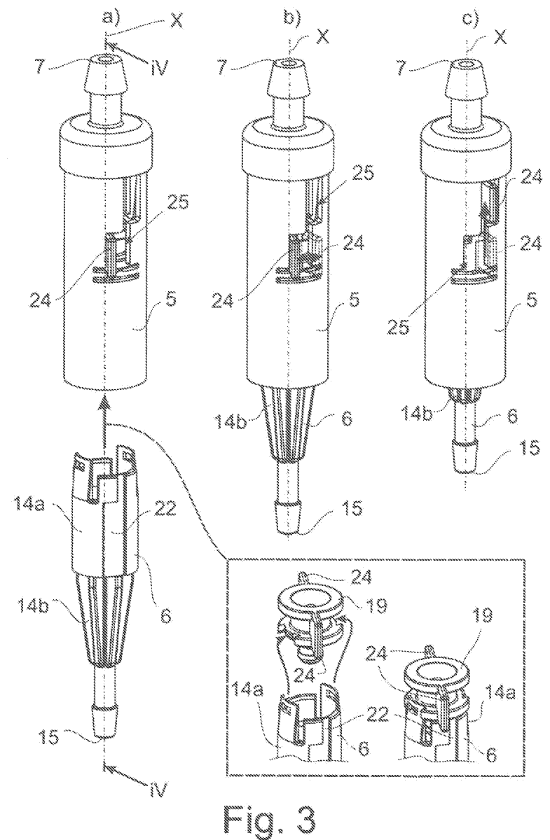

FIG. 3 in a perspective view, the two coupling devices of the sterile connector from FIG. 1, a) in the initial state before a coupling process, b) in the coupled state after the coupling process before an activation process, and c) in the coupled state after the activation process,

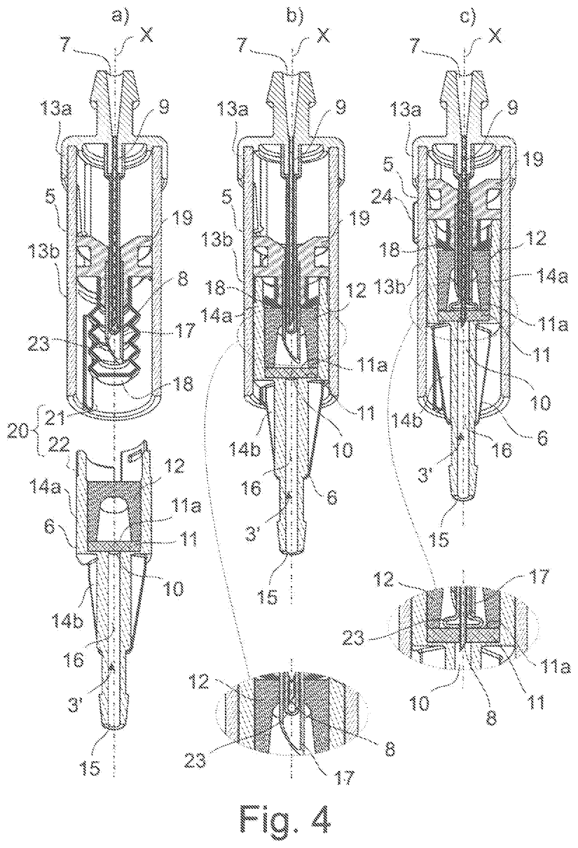

FIG. 4 in a sectional view, the two coupling devices of the sterile connector from FIG. 1, a) in the initial state before a coupling process, b) in the coupled state after the coupling process before an activation process, and c) in the coupled state after the activation process,

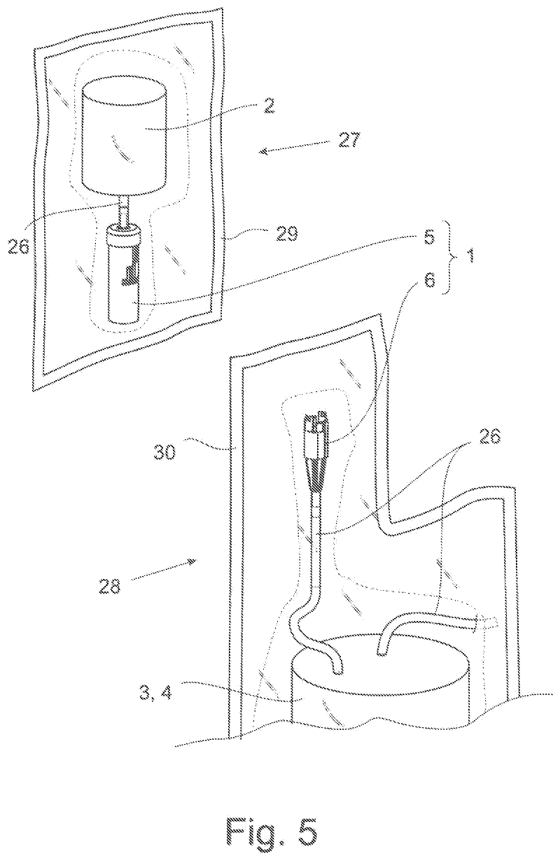

FIG. 5 in a perspective view, a packing arrangement as proposed with a first coupling device of the sterile connector as proposed according to FIG. 1, and a liquid container connected thereto, and a packing arrangement as proposed with the second coupling device of the sterile connector as proposed according to FIG. 1, and a bioprocess-technical system connected thereto,

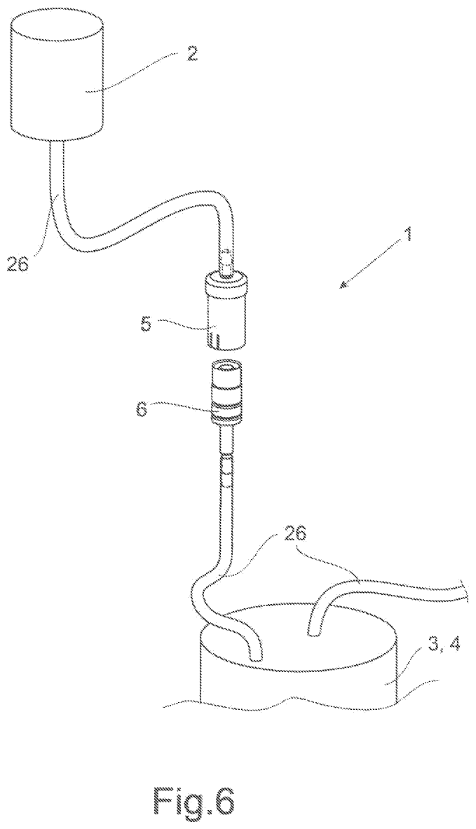

FIG. 6 in a perspective view, a sterile connector as proposed according to the second exemplary embodiment, during connection of its two coupling devices,

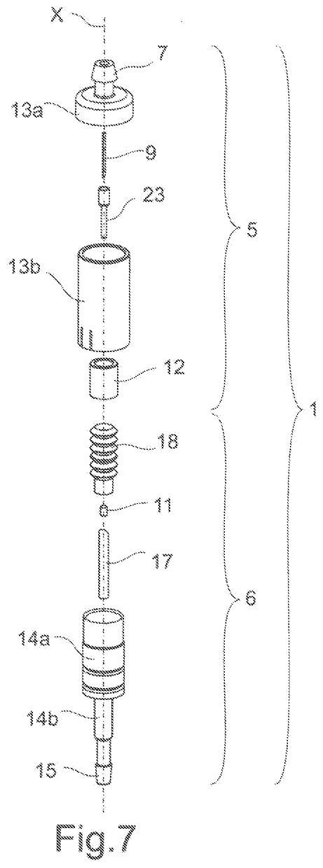

FIG. 7 in a perspective view, the sterile connector as proposed according to FIG. 6 with its two coupling devices, in exploded illustration,

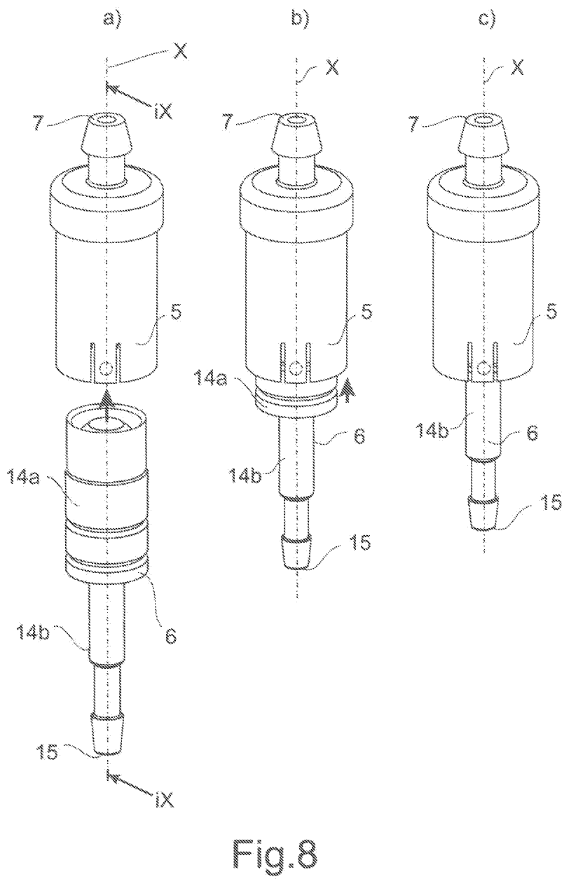

FIG. 8 in a perspective view, the two coupling devices of the sterile connector according to FIG. 6), a) in the initial state before a coupling process, b) in the coupled state after the coupling process before an activation process, and c) in the coupled state after the activation process,

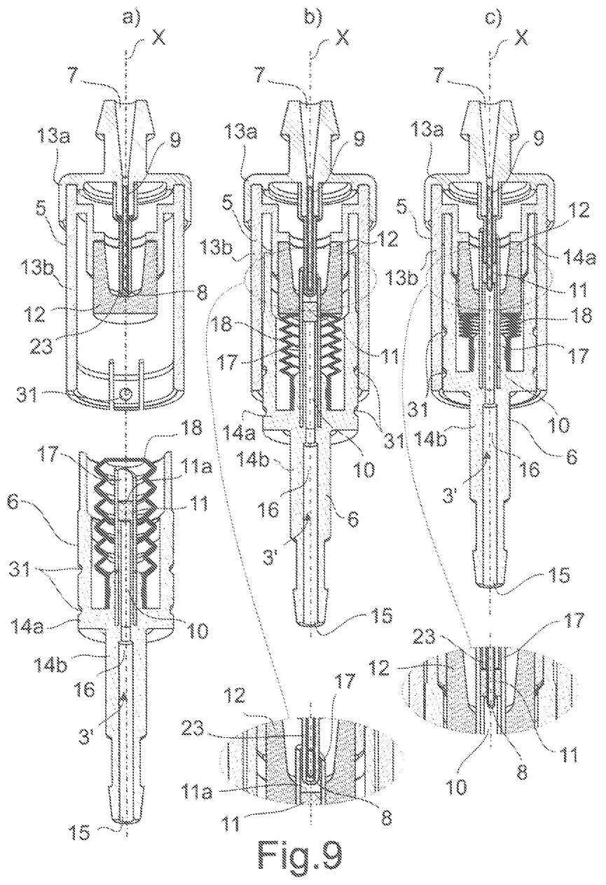

FIG. 9 in a sectional view, the two coupling devices of the sterile connector according to FIG. 6), a) in the initial state before a coupling process, b) in the coupled state after the coupling process before an activation process, and c) in the coupled state after the activation process,

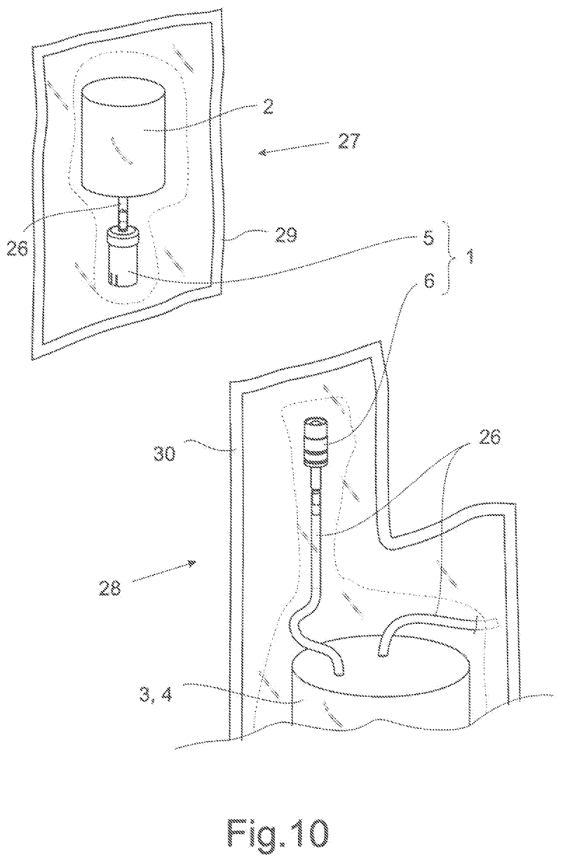

FIG. 10 in a perspective view, a packing arrangement as proposed with the first coupling device of the sterile connector as proposed according to FIG. 6, and a liquid container connected thereto, and a packing arrangement as proposed with the second coupling device of the sterile connector as proposed according to FIG. 6, and a bioprocess-technical system connected thereto.

The sterile connector 1 as proposed, shown in FIGS. 1 to 5, according to the first exemplary embodiment, and the sterile connector 1 as proposed, shown in FIGS. 6 to 10, according to the second exemplary embodiment, each serve for the sterile transfer of a liquid, in particular biological medium from a liquid container 2, here a small capacity liquid container, i.e. a liquid container with low capacity volume of for example maximum 30 ml, to a fluid chamber 3 which here and preferably is formed by a bioprocess-technical system 4, here a bioreactor. The sterile connector 1 as proposed has a first and a second coupling device 5, 6 which can be connected together mechanically and fluidically, and of which the first coupling device 5 is assigned to the liquid container 2 and the second coupling device 6 to the bioprocess-technical system 4. The term “assigned” means that the first coupling device 5 can be or is connected to the liquid container 2, and the second coupling device 6 can be or is connected to the bioprocess-technical system 4. Particularly preferably, firstly the liquid container 2 has been connected to its assigned first coupling device 5, and secondly the bioprocess-technical system 4 has been connected to its assigned second coupling device 6, by the manufacturer in sterile state.

The bioprocess-technical system 4, in particular the bioreactor, here forms a closed system or is integrated in a closed system. The bioreactor is for example designed as a laboratory bioreactor, i.e. as a bioreactor which, in contrast to a production bioreactor, has a relatively small working volume (maximum fill volume available) of maximum 10 liters. Such a bioreactor serves for performance of a biotechnological process, which here and preferably serves for production of advanced therapy medical products (ATMP). For example, the biotechnological process may also be a cell expansion process for T-cells. In any case, a biological reaction medium is provided in the bioreactor which in particular comprises tissue cells or microbial cells and a nutrient medium. Here, it is necessary to add the liquid media to the closed system in sterile state.

It is now essential that the first coupling device 5 has a fluid inlet 7 and a fluid outlet 8 fluidically connected thereto, wherein the first coupling device 5 has a first, preferably sterile cannula 9 which has an end facing away from the fluid inlet 7 and forming the fluid outlet 8; that the second coupling device 6 has a preferably sterile fluid passage 10 which in the initial state of the sterile connector 1 is covered axially at the front, i.e. away from the fluid passage 10, by the first septum 11; that in the initial state of the sterile connector 1, the fluid outlet 8 formed by the first cannula 9 is arranged in an in particular sterile region of the first coupling device 5 which is hermetically sealed against the environment of the first coupling device 5, and the surface 11a of the first septum 11 pointing away from the fluid passage 10 is arranged in an in particular sterile region of the second coupling device 6 which is hermetically sealed against the environment of the second coupling device 6; and that starting from the initial state of the sterile connector 1, in a fluidic connection process, a fluidic connection can be created between the fluid inlet 7 of the first coupling device 5 and the fluid passage 10 of the second coupling device 6, in that the end of the first cannula 9 forming the fluid outlet 8 in any case pierces the first septum 11 of the second coupling device 6.

A “septum”, also known as a puncture membrane, generally means a membrane separating two chamber portions from one another. It has the general effect that any adhesions on the outside of a cannula are stripped off during piercing of the septum. Preferably, it is made of a plastic material, further preferably an elastomer, in particular silicone material. Such a septum may have any form. In particular, a septum such as the first septum 11 here may be disk-like, or like the second septum 12 described below, may be pot-like. In the preferred case that—as here—a first septum 11 and a second septum 12 are provided, these may have different or the same forms (disk-like, pot-like), wherein preferably—as here—only one septum 11, 12, in particular the second septum 12, or alternatively (not shown here) both the first and the second septums 11, 12, is/are pot-like. The term “pot-like” means that a chamber portion is delimited by radial walls and an axial wall. The terms “axial” and “radial”, and the term “in the circumferential direction” as used below, here always relate to a longitudinal axis X of the sterile connector along which the two coupling devices 5, 6 extend during correct transfer of the liquid medium.

The first exemplary embodiment will now be described below.

As FIGS. 2 and 4 in particular illustrate, the first cannula 9 of the first coupling device 5 is arranged such that the liquid medium can flow from the fluid inlet 7 to the fluid outlet 8 via the first, preferably sterile cannula 9. Here and preferably, the first cannula 9, which is preferably made of metal, in particular stainless steel, is directly fluidically connected to the fluid inlet 7. The fluid inlet 7 is here formed by a housing part 13a, here a cap-like housing part 13a, of the first coupling device 5, which is preferably made of a thermoplastic, in particular PE (polyethylene). The fluid inlet 7 is here arranged centrally in the housing part 13a and/or coaxially to the longitudinal axis X of the sterile connector. The first cannula 9, which here also runs coaxially to the longitudinal axis X of the sterile connector and hence coaxially to the fluid inlet 7, is pushed into the housing part 13a and connected by substance bonding and/or axial force fit to the housing part 13a. The housing part 13a is here fixed by substance bonding and/or force fit to a housing part 13b of the first coupling device 5, in particular pushed onto the housing part 13b, which is preferably made of a thermoplastic, in particular PC (polycarbonate). The housing part 13b is here and preferably designed so as to be substantially cylindrical with a cylinder axis coaxial to the longitudinal axis X of the sterile connector.

The second coupling device 6 also has two housing parts 14a, 14b which are preferably made of a thermoplastic, in particular PC (polycarbonate), of which the housing part 14a is here and preferably also configured so as to be substantially cylindrical with a cylinder axis coaxial to the longitudinal axis X of the sterile connector. The first septum 11 and the second septum 12, to be described below, are arranged in the first housing part 14a, wherein the first septum 11 covers the preferably sterile fluid passage 10 axially towards the front, and thus here also the preferably sterile outlet channel 16, and thereby here and preferably hermetically closes the fluid passage 10. The term “axially towards the front” means in the direction from which the first coupling device 5 is supplied during the connection process, i.e. the direction towards the first coupling device 5.

The first septum 11 guarantees the sterility of the fluid passage 10 until this is punctured, as will be described below, in that it protects the fluid passage 10 from contact by the user or other components.

The second housing part 14b forms a fluid chamber 3′ which extends in particular along the longitudinal axis X of the sterile connector from the fluid passage 10 of the second coupling device 6 to the fluid outlet 15 of the second coupling device 6. The fluid passage 10 thus here too opens into a fluid chamber 3′ of the second coupling device 6 which has a fluid outlet 15 fluidically connected to the fluid passage 10, and to this extent forms an outlet channel 16. In an alternative embodiment (not shown here), the fluid chamber 3′ may also be otherwise closed, with the exception of the fluid passage 10, i.e. forming a further liquid container. In both alternatives, the second housing part 14b of the second coupling device 6 is connected in particular axially and rotationally fixedly to the first housing part 14a of the second coupling device 6, and preferably formed integrally.

During the fluidic connection process illustrated schematically in FIGS. 3 and 4, now the front end of the first cannula 9, which was formerly arranged in the hermetically sealed, in particular sterile region of the first coupling device 5 and forms the fluid outlet 8, is brought into contact with the first septum 11 which was formerly arranged in the hermetically sealed, in particular sterile region of the second coupling device 6, wherein then to create a fluidic connection, the front end of the first cannula 9 pierces the first septum 11 and thus the fluid outlet 8 penetrates the first septum 11. The fluidic connection process here comprises firstly a coupling process and secondly an activation process which will be described in more detail below.

The coupling process is illustrated in FIGS. 3a) and 4a). In the coupling process, the first coupling device 5 is mechanically connected to the second coupling device 6, which is depicted in FIG. 3a) by an arrow. The second coupling device 6 is here and preferably pushed into the first coupling device 5. In particular, for this the housing part 14a of the second coupling device 6 has a smaller cross-section than the housing part 13b of the first coupling device 5 into which the housing part 14a is introduced. This may however also be reversed, i.e. the second coupling device 6 may also be pushed onto the first coupling device 5, wherein then in particular the housing part 14a of the second coupling device 6 has a greater cross-section than the housing part 13b of the first coupling device 5 onto which the housing part 14a is pressed.

In this way, the sterile connector 1 is brought from the initial state shown in FIGS. 3a) and 4a) into the coupled state shown in FIGS. 3b) and 4b). The coupling process in particular comprises a linear movement, here and preferably exclusively a linear movement, of the first coupling device 5 relative to the second coupling device 6 along the longitudinal axis X of the sterile connector. In principle however, another movement or a combination of movements, for example a combined linear and rotational movement, in particular a screwing movement, of the first coupling device 5 relative to the second coupling device 6 is conceivable in order to perform the coupling process.

The coupling process is followed by an activation process which is illustrated firstly in FIGS. 3b) and 3c) and secondly in FIGS. 4b) and 4c). During the activation process, the first coupling device 5 is adjusted relative to the second coupling device 6 from a starting position (FIGS. 3b) and 4b)) to an operating position (FIGS. 3c) and 4c)), which is depicted in FIGS. 3b) and c) by arrows. This takes place in the coupled state, i.e. when the second coupling device 6 has been pushed into the first coupling device 5. In this way, the front end of the first cannula 9 of the first coupling device 5, forming the fluid outlet 8, pierces the first septum 11 of the second coupling device.

As FIGS. 3b) and 4b) show, the activation process here and preferably initially comprises a rotational movement of the first coupling device 5 relative to the second coupling device 6, namely in the circumferential direction around the longitudinal axis X of the sterile connector. The rotational movement here serves to adjust the coupling devices 5, 6 relative to one another from the starting position, into which the coupling devices 5, 6 have been brought by the coupling process, in particular the linear movement of the coupling process, into an intermediate position indicated in dotted lines in FIGS. 3b) and c). As FIGS. 3c) and 4c) show, the activation process here also comprises a linear movement of the first coupling device 5 relative to the second coupling device 6, also along the longitudinal axis X of the sterile connector. The linear movement, which here in particular follows the rotational movement, serves to adjust the coupling devices 5, 6 relative to one another from the intermediate position to the operating position. The latter movement, i.e. the linear movement, causes the piercing of the first septum 11 by the first cannula 9. Here and preferably, two individual movements are provided, namely the rotational movement firstly and the linear movement secondly, wherein however in principle it is also conceivable that only a single movement is performed during the activation process, for example solely a linear movement, or a combined linear and rotational movement, in particular a screwing movement.

Now the individual elements, which cooperate with one another, of the first coupling device 5 and the second coupling device 6 of the first exemplary embodiment and their function will be explained in more detail.

In the initial state, as FIG. 4a) shows, the hermetically sealed, preferably sterile region of the second coupling device 6 is in particular limited at least partially, here and preferably completely, by the first septum 11 and the second septum 12. In principle however, it is also conceivable that the hermetically sealed region of the second coupling device 6 is limited not only by the first septum 11 and the second septum 12, but also by a wall portion of the second coupling device 6, and in particular a wall portion of the housing part 14a. The essential factor is only that the first septum 11 and the second septum 12 hermetically seal the chamber portion formed between them.

Since the second septum 12 protects the first septum 11 from contact by the user or other components, this guarantees the sterility of the first septum 11 until the coupling process, and in particular until the second septum 12 is pierced, as will be described below.

The coupling process, in particular the linear movement of the coupling process, as shown in FIG. 4b), now creates an access for the first cannula 9 through the second septum 12 into the preferably sterile region of the second coupling device 6 which is hermetically sealed in the initial state, and/or the end of the first, preferably sterile cannula 9 forming the fluid outlet 8 protrudes axially into the region of the second coupling device 6 which is hermetically sealed in the initial state. To this end, the first coupling device 5 here and preferably comprises a second, preferably sterile cannula 17 which is arranged coaxially to the first cannula 9 and through which the first cannula 9 can be moved. The second cannula 17, which is preferably made of metal, in particular brass, to this end here has a greater cross-section than the first cannula 9. During the activation process, the first cannula 9, as shown in FIG. 4c), can be moved so far through the second cannula 17 that the cannula end of the first cannula 9 forming the fluid outlet 8 protrudes out of the second cannula 17 and can touch and pierce the first septum 11. The term “move through” as used here means in general that the first cannula 9 is axially displaced relative to the second cannula 17, wherein this is not—or in any case not necessarily—caused by a movement of the first cannula 9 inside the housing part 13a, 13b, but rather is caused by a linear movement of the second cannula 17 inside the housing part 13b along the longitudinal axis X of the sterile connector. Preferably therefore, only the second cannula 17 is actively movable inside the first coupling device 5, and not the first cannula 9.

The coupling process, in particular the linear movement of the coupling process, now causes a cannula portion of the second cannula 17 to pierce the second septum 12, and thus create the access for the first cannula 9 into the preferably sterile region of the second coupling device 6 which is hermetically sealed in the initial state, and/or creates an access for the first cannula 9 to the first septum 11. Here it is provided that the first cannula 9 and the second septum 12 do not touch one another before, during and after the coupling process. Contamination of the first cannula 9 by the second septum 12 can thus be excluded.

In order to delimit the hermetically sealed, preferably sterile region of the first coupling device 5 from the environment, the first coupling device 5 has at least one sealing envelope. Thus initially a first sealing envelope 18 is provided, here and preferably in the form of a bellows, which in the initial state radially and at the front end surrounds the axial cannula portion of the second cannula 17 which pierces the second septum 12 during the coupling process. Here, in the initial state, the first sealing envelope 18 or the bellows, as shown in FIG. 4a), is axially closed towards a side facing the second coupling device 6 during the coupling process, but open towards the opposite side, i.e. towards the first cannula 9. The first sealing envelope 18 is preferably made of a silicone material.

The first sealing envelope 18 is here and preferably fixed to a cannula holder 19 for the second cooler 17, which will be described in more detail below. The first sealing envelope 18 is hermetically tightly connected to the cannula holder 19. According to an alternative embodiment (not shown here), it is also conceivable that the first sealing envelope 18 lies hermetically tightly on and/or is fixed to an axial cannula portion of the second cannula 17 and/or a wall portion of the first coupling device, in particular of the housing part 13b.

The first sealing envelope 18 guarantees the sterility of the axial cannula portion of the second cannula 17 until penetration into the second septum 12, in that it protects the second cannula 17 from contact by the user or other components. The second cannula 17 pierces the first sealing envelope 18 preferably only during the coupling process, when the first sealing envelope 18 comes to rest axially on the second septum 12. Preferably, the second cannula 17 penetrates into the second septum 12 immediately after piercing the first sealing envelope 18, and then pierces this septum completely.

In the case when, as shown in FIG. 4b), the coupling process, in particular the linear movement of the coupling process, causes the axial cannula portion of the second cannula 17 to pierce the first sealing envelope 18, preferably the first cannula 9 and the first sealing envelope 18 do not touch one another before, during and/or after the coupling process. In this way, contamination of the first cannula 9 by the first sealing envelope 18 is excluded.

As stated, in the exemplary embodiment depicted here and to this extent preferred, the second cannula 17 is fixed to a cannula holder 19 which is preferably made of a thermoplastic, in particular PC (polycarbonate). The cannula holder 19 is mounted so as to be axially movable in the first coupling device 5. Because of the coupling process here, now the second coupling device 6, in particular the housing part 14a, comes to rest axially on the cannula holder 19. Particularly preferably, the coupling process creates the axial and/or radial connection of the second coupling device 6 or the housing part 14a to the cannula holder 19 by form fit or force fit. In the exemplary embodiment shown here, the second coupling device 6 or the housing part 14a is latched onto the cannula holder 19. Thus an axial and/or radial displacement of the cannula holder 19 in the first coupling device 5, or relative to the housing part 13b, causes the relative movement of the first cannula 9 inside the second cannula 17, and finally the piercing of the first septum 11 by the first cannula 9 during the activation process.

In order to guarantee a defined relative movement between the first coupling device 5 and the second coupling device 6 in the coupling process, here and preferably also a guide device 20 is provided. This achieves axial guidance of the first coupling device 5 relative to the second coupling device 6 during the coupling process. Thus a protrusion or axially running web 21 and/or an axially running groove may be provided on a wall portion of the first coupling device 5, in particular on the housing part 13b, preferably on the inside, and a corresponding counter-piece 22 provided on a wall portion of the second coupling device 6, in particular on the housing part 14a, preferably on the outside. The counter-piece 22 is for example an axially running groove corresponding to the protrusion or axially running web 21 of the first coupling device 5, or a protrusion or axially running web corresponding to the axially running groove of the first coupling device 5. The protrusion or web 21 or the groove of the first coupling device 5, and the corresponding counter-piece 22 of the second coupling device 6, then together form the guide device 20. In the exemplary embodiment shown here, two such guide devices 20 are provided opposite one another on the radial sides of the sterile connector 1.

As FIG. 3a) shows, the respective guide device 20 here also defines the angular position or positions in the circumferential direction in which the first coupling device 5 must be connected to, in particular plugged together with, the second coupling device 6 for the coupling process. Since here two guide devices 20 are provided on opposite radial sides of the sterile connector 1 and here arranged at an angular spacing of 180° to one another in the circumferential direction, the first coupling device 5 can be connected to the second coupling device 6 in two different angular positions. It is however also conceivable to provide only a single guide device 20, or two or more guide devices 20, and in particular arrange these such that the first coupling device 5 can be connected to the second coupling device 6 only in a single angular position.

As FIGS. 4a) to c) show, the first coupling device 5 furthermore has a second sealing envelope 23, in particular in the form of a hose closed at one end, which in the initial state, radially and at the front end forming the fluid outlet 8, surrounds an axial cannula portion of the first cannula 9 having the fluid outlet 8 which pierces the first septum 11 during the activation process. The second sealing envelope 23 is preferably made of a silicone material. The second sealing envelope 23 surrounds the first cannula 9 here and preferably over the majority of its length or substantially its entire length. In the initial state, the second sealing envelope 23 is axially closed towards the side facing the second coupling device 6 during the coupling process, but open on the side facing the fluid inlet 7. With the closed side then, in the activation process, the first cannula 9 pierces the second sealing envelope 23 before the first cannula 9 penetrates into the first septum 11. In the initial state, the second sealing envelope 23 here and preferably also rests on the inside of the second cannula 17, whereby the hermetically sealed region surrounded by the first sealing envelope 18 is also hermetically sealed through the second cannula 17.

The second sealing envelope 23 is here and preferably fixed to the housing part 13a. The second sealing envelope 23 is hermetically tightly connected to the housing part 13a. According to an alternative embodiment (not shown here), it is also conceivable that the second sealing envelope 23 rests hermetically tightly against and/or is fixed to an axial cannula portion of the first cannula 9 and/or the housing part 13b.

This second sealing envelope 23 guarantees the sterility of the axial cannula portion of the first cannula 9 surrounding the fluid outlet 8 until penetration of the first cannula 9 into the first septum 11, in that it protects the first cannula 9 from contact by the user or other components. The first cannula 9 preferably only pierces the second sealing envelope 23 when the latter rests axially on the first septum 11. Preferably immediately after piercing the second sealing envelope 23, the first cannula 9 penetrates into the septum 11.

In order to be able to perform the activation process manually after the coupling process, which mechanically connects the first coupling device 5 to the second coupling device 6, here and preferably an actuation element 24 is provided on the cannula holder 19 for manual actuation of the cannula holder 19. By means of the actuation element 24, the cannula holder 19 together with the second cannula 17 and the second coupling device 6 can be moved generally linearly and here also rotationally relative to the first coupling device 5 during the activation process. Here and preferably, two such actuation elements 24 are provided on opposite radial sides of the sterile connector 1 or first coupling device 5 or cannula holder 19. Here and preferably, the actuation element 24 extends radially outward from the cannula holder 19 through a guide opening 25 which is provided in a wall portion of the first coupling device 5, in particular in the housing part 13b.

The actuation element 24 is here and preferably mounted in the guide opening 25 so as to be axially and here also rotationally displaceable during the activation process. Here, during the activation process, the actuation element 24 is displaceable in the guide opening 25 initially in the circumferential direction (FIGS. 3b and 4b)) and then axially (FIGS. 3c) and 4c)). The displacement movement of the actuation element 24 in the guide opening 25, depicted by an arrow in FIGS. 3b) and c), is here and preferably limited by the edge of the guide opening 25 so that the user also receives feedback indicating the current position of the coupling devices 5, 6 relative to one another.

The illustration of the actuation element 24 in dotted lines shows an intermediate position into which the actuation element 24 is moved by its displacement in the circumferential direction, and out of which then the actuation element 24 is moved linearly. The solid lines indicate the respective end positions of the actuation element 24 in the guide opening 25, wherein FIG. 3b) shows a first end position which correlates with the starting position of the two coupling devices 5, 6, and FIG. 3c) shows a second end position which correlates with the operating position of the two coupling devices 5, 6.

FIG. 3b) furthermore shows a possibility that the actuation element 24 is latched in the guide opening 25 by the activation process when the guide element 24 is in its second end position. This prevents an unintentional deactivation of the sterile connector 1 after a continuous fluidic connection has been created in the sterile connector 1 in the activation process.

It should be pointed out that FIGS. 3 and 4 show the coupling devices 5, 6 in unattached state for reasons of clarity. In fact, with the sterile connector 1 shown in the present exemplary embodiment, the fluid inlet 7 of the first coupling device 5 and the fluid outlet 15 of the second coupling device 6 can be or are connected, in particular via a plug connection, to a hose 26 or tube as depicted in FIG. 1. Preferably, the fluid inlet 7 of the first coupling device 5 is coupled via a hose 26 or tube to the liquid container 2. In addition or alternatively, the fluid outlet 15 of the second coupling device 6 may be fluidically connected via a hose 26 or tube to the fluid chamber 3 of the bioprocess-technical system 4, in particular the bioreactor.

Now the second exemplary embodiment will be explained in more detail.

As FIGS. 7 and 9 in particular illustrate, the first cannula 9 of the first coupling device 5 is arranged such that the liquid medium can flow from the fluid inlet 7 via the first cannula 9 to the fluid outlet 8. Here and preferably, for this the first cannula 9 is fluidically connected directly to the fluid inlet 7. The fluid inlet 7 is here formed by a housing part 13a, here a cap-like housing part 13a, of the first coupling device 5. Here, the fluid inlet 7 is arranged centrally in the housing part 13a and/or coaxially to the longitudinal axis X of the sterile connector. The first cannula 9, which here also runs coaxially to the longitudinal axis X of the sterile connector and hence coaxially to the fluid inlet 7, is pushed into the housing part 13a and connected to the housing part 13a by substance bonding and/or axial force fit. The housing part 13a is here fixed to a housing part 13b of the first coupling device 5 by substance bonding and/or force fit, in particular pushed onto the housing part 13b. The housing part 13b is here and preferably designed so as to be substantially cylindrical with a cylinder axis coaxial to the longitudinal axis X of the sterile connector.

The second coupling device 6 also has two housing parts 14a, 14b, of which the housing part 14a is here and preferably also configured so as to be substantially cylindrical with a cylinder axis coaxial to the longitudinal axis X of the sterile connector. The first septum 11 and a first sealing envelope 18—to be described in more detail below—are arranged in the first housing part 14a, wherein the first septum 11 covers the fluid passage 10 and hence here also the outlet channel 16 axially to the front, and thereby here and preferably hermetically closes the fluid passage 10. The term “axially to the front” means in the direction from which the first coupling device 5 is supplied during the connection process, i.e. the direction towards the first coupling device 5.

The first septum 11 guarantees the sterility of the fluid passage 10 until this is pierced, as will be described below, in that it protects the fluid passage 10 from contact by the user or other components.

The second housing part 14b forms a fluid chamber 3′ which extends in particular along the longitudinal axis X of the sterile connector, from the fluid passage 10 of the second coupling device 6, to a fluid outlet 15 of the second coupling device 6. The fluid passage 10 thus here opens into a fluid chamber 3′ of the second coupling device 6 which has a fluid outlet 15 fluidically connected to the fluid passage 10, and to this extent forms an outlet channel 16. In an alternative embodiment (not shown here), the fluid chamber 3′ may also be otherwise closed with the exception of the fluid passage 10, i.e. forming a further liquid container. In both alternatives, the second housing part 14b of the second coupling device 6 is connected in particular axially and rotationally fixedly to the first housing part 14a of the second coupling device 6, and preferably formed integrally.

The fluidic connection process illustrated schematically in FIGS. 8 and 9 now brings the front end of the first cannula 9, which was formerly arranged in the hermetically sealed, in particular sterile region of the first coupling device 5 and forms the fluid outlet 8, into contact with the first septum 11 which was formerly arranged in the hermetically sealed, in particular sterile region of the second coupling device 6, wherein then to create a fluidic connection, the front end of the first cannula 9 pierces the first septum 11 and thus the fluid outlet 8 penetrates the first septum 11. The fluidic connection process here comprises firstly a coupling process and secondly an activation process which will be described in more detail below.

The coupling process is illustrated in FIGS. 8a) and 9a). In the coupling process, the first coupling device 5 is mechanically connected to the second coupling device 6, which is depicted in FIG. 8a) by an arrow. The second coupling device 6 is here and preferably pushed into the first coupling device 5. In particular, for this the housing part 14a of the second coupling device 6 has a smaller cross-section than the housing part 13b of the first coupling device 5 in which the housing part 14a is introduced. This may however also be reversed, i.e. the second coupling device 6 may also be pushed onto the first coupling device 5, wherein then in particular the housing part 14a of the second coupling device 6 has a greater cross-section than the housing part 13b of the first coupling device 5 onto which the housing part 14a is pressed.

In this way, the sterile connector 1 is brought from the initial state shown in FIGS. 8a) and 9a) into the coupled state shown in FIGS. 8b) and 9b). The coupling process in particular comprises a linear movement, here and preferably exclusively a linear movement, of the first coupling device 5 relative to the second coupling device 6 along the longitudinal axis X of the sterile connector. In principle however, another movement or a combination of movements, for example a combined linear and rotational movement, in particular a screwing movement, of the first coupling device 5 relative to the second coupling device 6 is conceivable in order to perform the coupling process.

The coupling process is followed by an activation process which is illustrated firstly in FIGS. 8b) and 8c) and secondly in FIGS. 9b) and 9c). During the activation process, the first coupling device 5 is adjusted relative to the second coupling device 6 from a starting position (FIGS. 8b) and 9b)) to an operating position (FIGS. 8c) and 8c)), which is depicted in FIGS. 8b) and c) by arrows. This takes place in the coupled state, i.e. when the second coupling device 6 has been pushed into the first coupling device 5. In this way, the front end of the first cannula 9 of the first coupling device 5, forming the fluid outlet 8, pierces the first septum 11 of the second coupling device 6.

As FIGS. 8b) and 9b) show, the activation process here comprises in particular a linear movement, here exclusively a linear movement, of the first coupling device 5 relative to the second coupling device 6, namely parallel to the longitudinal axis X of the sterile connector and in particular coaxially to the linear movement of the coupling process. The linear movement here serves to adjust the coupling devices 5, 6 relative to one another from the starting position, in which the coupling devices 5, 6 are brought by the coupling process, in particular the linear movement of the coupling process, into an operating position. This movement causes the piercing of the first septum 11 by the first cannula 9. In principle, it is also conceivable that, instead of a purely linear movement, a combined linear and rotational movement is provided for bringing the coupling devices 5, 6 from the initial position to the operating position during the activation process.

Now the individual elements, which cooperate with one another, of the first coupling device 5 and second coupling device 6 of the second exemplary embodiment, and their function will be explained in more detail.

In the initial state, as FIG. 9a) shows, the hermetically sealed and preferably sterile region of the second coupling device 6 is in particular limited at least partially, here and preferably completely, by the first septum 11 and the first sealing envelope 18 which here has the form of a bellows.

Since the first sealing envelope 18 protects the first septum 11 and a second cannula 17 (presented in more detail below), in which the first septum 11 is arranged in particular axially fixedly, from contact by the user or other components, the first sealing envelope 18 guarantees the sterility of the first septum 11 and the second cannula 17 until the coupling process.

The coupling process, in particular the linear movement of the coupling process, as shown in FIG. 9b), now forms an access for the first, preferably sterile cannula 9 in the region of the second coupling device 6 which is hermetically sealed in the initial state, and/or the end of the first, preferably sterile cannula 9 forming the fluid outlet 8 protrudes axially into the region of the second coupling device 6 which is hermetically sealed in the initial state. To this end, the second coupling device 6 here and preferably already comprises said second, preferably sterile cannula 17 into which the first cannula 9 can be moved during the coupling process and which is then arranged coaxially to the first cannula 9 in the coupled state. To this end, the second cannula 17 here has a greater cross-section than the first cannula 9. During the activation process, the first cannula 9, as shown in FIG. 9c), can be moved so far into the second cannula 17 that the cannula end of the first cannula 9 forming the fluid outlet 8 can touch and pierce the first septum 11.

In the exemplary embodiment shown here and to this extent also preferred, the second cannula 17 is connected axially fixedly and in particular rotationally fixedly to the housing part 14a of the second coupling device 6.

In order for the first cannula 9 of the first coupling device 5 to be able to come into contact with the first septum 11 of the second coupling device 6, firstly an access thus be created through a second septum 12 in the first coupling device 5. This is achieved in that because of the coupling process, in particular the linear movement of the coupling process, an axial cannula portion of the second cannula 17 of the second coupling device 6 pierces the second septum 12 of the first coupling device 5, and thus creates an access for the first cannula 9 in the region of the second coupling device 6 which is hermetically sealed in the initial state, and/or an access for the first cannula 9 to the first septum 11. It is provided here that the first cannula 9 and the second septum 12 do not touch one another before, during and/or after the coupling process. Contamination of the first cannula 9 by the second septum 12 can thus be excluded.

In order to delimit the hermetically sealed, preferably sterile region of the first coupling device 5 against the environment in the initial state of the sterile connector 1, the first coupling device 5 comprises, as well as the second septum 12, here also a second sealing envelope 23, in particular in the form of a hose closed at one end, which surrounds the first cannula 9 radially and at the front end forming the fluid outlet 8.

The first sealing envelope 18 is here and preferably fixed to the housing part 14a. Here, the first sealing envelope 18 is hermetically tightly connected to the housing part 14a. According to an alternative embodiment (not shown here), it is also conceivable that the first sealing envelope 18 lies hermetically tightly against and/or is fixed to an axial cannula portion of the second cannula 17.

The first sealing envelope 18 guarantees the sterility of the axial cannula portion of the second cannula 17 until its penetration into the second septum 12, in that it protects the second cannula 17 from contact by the user or other components. The second cannula 17 pierces the first sealing envelope 18 preferably only during the coupling process, when the first sealing envelope 18 comes to rest axially on the second septum 12. Preferably, the second cannula 17 penetrates into the second septum 12 immediately after piercing of the first sealing envelope 18, and then completely pierces said septum.

In the case when, as shown in FIG. 9b), the coupling process, in particular the linear movement of the coupling process, causes the axial cannula portion of the second cannula 17 to pierce the first sealing envelope 18, preferably the first cannula 9 and the first sealing envelope 18 do not touch one another before, during and/or after the coupling process. In this way, contamination of the first cannula 9 by the first sealing envelope 18 is also excluded.

As FIGS. 9a) to c) show, the first coupling device 5 furthermore comprises said second sealing envelope 23, which in the initial state, radially surrounds an axial cannula portion of the first cannula 9 having the fluid outlet 8 which pierces the first septum 11 during the activation process. The second sealing envelope 23 surrounds the first cannula 9 here and preferably over the majority of its length or substantially its entire length. In the initial state, the second sealing envelope 23 is axially closed towards the side facing the second coupling device 6 during the coupling process, but open on the side facing the fluid inlet 7. With the closed side then, the first cannula 9 in the activation process pierces the second sealing envelope 23 before the first cannula 9 penetrates into the first septum 11.

The second sealing envelope 23 is here and preferably fixed to the housing part 13a. The second sealing envelope 23 is hermetically tightly connected to the housing part 13a. According to an alternative embodiment (not shown here), it is also conceivable that the second sealing envelope 23 rests hermetically tightly against and/or is fixed to an axial cannula portion of the first cannula 9 and/or the housing part 13b.

This second sealing envelope 23 guarantees the sterility of the axial annular portion of the first cannula 9 surrounding the fluid outlet 8 until penetration of the first cannula 9 into the first septum 11, in that it protects the first cannula 9 from contact by the user or other components. The first cannula 9 preferably only pierces the second sealing envelope 23 when this rests axially on the first septum 11. Preferably immediately after piercing the second sealing envelope 23, the first cannula 9 penetrates into the septum 11.

Finally, here and preferably, as a combined view of FIGS. 9a) to c) shows, a mechanism 31 is provided for indication, in particular haptic indication, of at least one defined axial position of the first coupling device 5 relative to the second coupling device 6. Preferably, in the respective defined axial position, in particular via a snap-fit connection, an axial form fit is created between the first coupling device 5 and the second coupling device 6. The snap-fit connection may be released again on overcoming a defined axial force which is applied between the two coupling devices 5, 6. A defined axial position in particular corresponds to the starting position in the coupled state (FIGS. 8b) and 9b)). Furthermore, a defined axial position here corresponds to the operating position in the coupled state (FIGS. 8c) and 9c)), i.e. the state after completion of the activation process. The first coupling device 5 may here be brought from the initial state into the respective defined axial position relative to the second coupling device 6. In this way, the user detects, in particular haptically, the reaching of the starting position and/or the operating position.

It is pointed out that in FIGS. 8 and 9, the coupling devices 5, 6 are depicted in unattached state for reasons of clarity. In fact, with the sterile connector 1 shown in the present exemplary embodiment, the fluid inlet 7 of the first coupling device 5 and the fluid outlet 15 of the second coupling device 6 can be or are connected, in particular via a plug connection, to a hose 26 or tube as depicted in FIG. 6. Preferably, the fluid inlet 7 of the first coupling device 5 is coupled via a hose 26 or tube to the liquid container 2. In addition or alternatively, the fluid outlet 15 of the second coupling device 6 may be fluidically connected via a hose 26 or tube to the fluid chamber 3 of the bioprocess-technical system 4, in particular the bioreactor.

According to a further teaching which has independent significance, a packing arrangement 27, 28 is claimed with a packing 29, 30 and at least one, preferably precisely one first or second coupling device 5, 6, packed in sterile fashion therein, of a sterile connector 1 as proposed, or with a sterile connector 1 as proposed packed in sterile fashion therein (FIGS. 5 and 10). Reference may therefore be made to all statements relating to the sterile connector 1 as proposed.

According to one exemplary embodiment, a packing arrangement 27 is proposed, as shown at the top in FIG. 5 and FIG. 10, with a packing 29 and with a first coupling device 5, packed in sterile fashion therein, of the sterile connector 1 as proposed, and with a liquid container 2, in particular filled, here already filled by the manufacturer, packed in sterile fashion therein. The liquid container 2 is here fluidically connected to the coupling device 5 of the sterile connector 1, i.e. already in the packed state, preferably via a hose 26 or a tube, wherein the coupling device 5 is not however yet connected to the further coupling device 6 of the sterile connector 1.

According to a further exemplary embodiment, a packing arrangement 28, as shown at the bottom in FIG. 5 and FIG. 10, is provided with a packing 30 and with the second coupling device 6, packed in sterile fashion therein, of the sterile connector 1 as proposed, and with a bioprocess-technical system 4, here a bioreactor, packed in sterile fashion therein. The bioprocess-technical system 4 or bioreactor is here fluidically coupled to the second coupling device 6 of the sterile connector 1, i.e. already in packed state, preferably via a hose 26 or a tube, wherein the coupling device 6 is however not yet coupled to the further coupling device 5 of the sterile connector 1.

In an alternative exemplary embodiment (not shown here), a packing arrangement is conceivable with a packing in which only a sterile connector 1 as proposed, comprising the two coupling devices 5, 6, is packed in sterile fashion. In principle, it is also conceivable to provide a packing arrangement in which only the first coupling device 5 or the second coupling device 6 of the sterile connector 1 as proposed is packed.

According to yet a further teaching which also has independent significance, a use is claimed of a sterile connector 1 as proposed, packed in sterile fashion, a first or second coupling device 5, 6, packed in sterile fashion, of a sterile connector 1 as proposed, and/or a packing arrangement 27, 28 as proposed, for the sterile transfer of a liquid, in particular biological medium, from a liquid container 2 to a fluid chamber 3, in particular of a bioprocess-technical system 4, for example a bioreactor. Also with respect to the claimed use, to this extent reference may be made to all statements relating to the sterile connector 1 as proposed and to the packing arrangement 27, 28 as proposed.

According to the packing arrangements 27, 28 shown in FIGS. 5 and 10, after unpacking of the unit of the first coupling device 5 and liquid container 2 on one side, and after unpacking of the unit of the second coupling device 6 and bioprocess-technical system 4 on the other, each together with the hoses 26 or tubes creating the connection, the first coupling device 5 is mechanically connected to the second coupling device 6 in a coupling process as described. Then a fluidic connection extending from the fluid inlet 7 of the first coupling device 5 to the fluid outlet 15 of the second coupling device 6 is created by the activation process as described, so that then the liquid medium may be transferred from the liquid container 2 via the first coupling device 5 and second coupling device 6, into the fluid chamber 3′ of the second coupling device 6, and/or then into the fluid chamber 3 or bioprocess-technical system 4, in particular the bioreactor.

In a particularly preferred embodiment, at least the first coupling device 5 or second coupling device 6 of the sterile connector 1 as proposed, in particular both coupling devices 5, 6, preferably also the liquid container 2 and/or the bioprocess-technical system 4 and/or the hose(s) 26 or tube(s), are each single-use components. The respective components, i.e. the respective coupling device 5, 6, the liquid container 2, the bioprocess-technical system 4 and/or the respective hose 26 or tube, are made at least partly and preferably at least mainly of a plastic material. Suitable plastics for the individual components are in particular a silicone material and/or a polymer material, in particular an elastomer and/or thermoplastic. Examples of these are PE (polyethylene), PP (polypropylene), PTFE (polytetrafluoroethylene), PBT (polybutylene terephthalate), PSU (polysulfone), PESU (polyethersulfone) or PC (polycarbonate).

1. A sterile connector for the sterile transfer of a liquid, in particular a biological medium, from a liquid container (2) into a fluid chamber (3, 3′), for example of a bioprocess-technical system (4), in particular of a bioreactor, wherein the sterile connector (1) has a first and a second coupling device (5, 6),
characterized in that
the first coupling device (5) has a fluid inlet (7) and a fluid outlet (8) fluidically connected thereto, wherein the first coupling device (5) comprises a first cannula (9) having an end which faces away from the fluid inlet (7) and forms the fluid outlet (8);
the second coupling device (6) has a fluid passage (10) which, in an initial state of the sterile connector (1), is axially covered by a first septum (11);
in the initial state of the sterile connector (1), the fluid outlet (8) formed by the first cannula (9) is arranged in an in particular sterile region of the first coupling device (5) which is hermetically sealed against the environment of the first coupling device (5), and the surface (11a) of the first septum (11) axially facing away from the fluid passage (10) is arranged in an in particular sterile region of the second coupling device (6) which is hermetically sealed against the environment of the second coupling device (6); and
in a fluidic connection process, starting from the initial state of the sterile connector (1), a fluidic connection can be created between the fluid inlet (7) of the first coupling device (5) and the fluid passage (10) of the second coupling device (6) in that the end of the first cannula (9) forming the fluid outlet (8) in any case pierces the first septum (11) of the second coupling device (6).

2. The sterile connector as claimed in claim 1, characterized in that the fluidic connection process is a coupling process in which the first coupling device (5) is mechanically connected to the second coupling device (6), in particular the second coupling device (6) is plugged into the first coupling device (5), and thereby the sterile connector (1) is brought from an initial state into a coupled state; preferably, the coupling process comprises a linear movement, in particular exclusively a linear movement, of the first coupling device (5) relative to the second coupling device (6).

3. The sterile connector as claimed in claim 1 or 2, characterized in that in coupled state, the fluidic connection process comprises an activation process in which the first coupling device (5) is adjusted relative to the second coupling device (6) from a starting position into an operating position, in particular when the second coupling device (6) is plugged into the first coupling device (5), and thereby the fluid outlet (8) of the first coupling device (5) pierces the first septum (11); preferably, the activation process comprises a linear movement, in particular also a rotational movement, of the first coupling device (5) relative to the second coupling device (6).

4. The sterile connector as claimed in any of the preceding claims, characterized in that the region of the second coupling device (6), which in the initial state is hermetically sealed, is delimited at least partially, preferably completely, by the first septum (11) and a second septum (12); preferably, the coupling process provides an access for the first cannula (9) through the second septum (12) into the region of the second coupling device (6) which is hermetically sealed in the initial state; and/or the end of the first cannula (9) forming the fluid outlet (8) protrudes axially into the region of the second coupling device (6) which is hermetically sealed in the initial state.

5. The sterile connector as claimed in any of the preceding claims, characterized in that the first coupling device (5) has a second cannula (17) which is arranged coaxially to the first cannula (9) and through which the first cannula (9) can be moved; preferably, the coupling process causes an axial cannula portion of the second cannula (17) to pierce the second septum (12) and thereby provide the access for the first cannula (9) into the region of the second coupling device (6) which is hermetically sealed in the initial state, and/or an access for the first cannula (9) to the first septum (11); furthermore preferably, the first cannula (9) and the second septum (12) do not touch one another before, during and/or after the coupling process.

6. The sterile connector as claimed in any of the preceding claims, characterized in that the region of the first coupling device (5), which is hermetically sealed in the initial state of the sterile connector (1), is delimited towards the environment at least partially by at least one sealing envelope (18, 23) of the first coupling device (5).

7. The sterile connector as claimed in any of the preceding claims, characterized in that the first coupling device (5) comprises a first sealing envelope (18), in particular in the form of a bellows, which in the initial state radially surrounds the axial cannula portion of the second cannula (17) which pierces the second septum (12) during the coupling process; wherein the first sealing envelope (18) in the initial state is axially closed towards a side facing the second coupling device (6) during the coupling process.

8. The sterile connector as claimed in any of the preceding claims, characterized in that the coupling process causes an axial cannula portion of the second cannula (17) to pierce the first sealing envelope (18); preferably, the first cannula (9) and the first sealing envelope (18) do not touch one another before, during and/or after the coupling process.

9. The sterile connector as claimed in any of the preceding claims, characterized in that the second cannula (17) is fixed to a cannula holder (19) which is mounted so as to be axially movable in the first coupling device (5); preferably, the coupling process brings the second coupling device (6) to rest axially on the cannula holder (19); further preferably, the coupling process causes the second coupling device (6) to be axially and/or radially connected with the cannula holder (19) by form fit or force fit; further preferably, the coupling process causes the second coupling device (6) to latch onto the cannula holder (19).

10. The sterile connector as claimed in any of the preceding claims, characterized in that a guide device (20) is provided which, during the coupling process, causes an axial guiding of the first coupling device (5) relative to the second coupling device (6); preferably, as a guide device (20), a protrusion or axially running web (21) and/or an axially running groove is provided on a wall portion of the first coupling device (5), in particular on the inside, and a corresponding counter-piece (22) is provided on a wall portion of the second coupling device (6), in particular on the outside.

11. The sterile connector as claimed in any of the preceding claims, characterized in that the first coupling device (5) comprises a second sealing envelope (23), in particular in the form of a hose which is closed at one end, which in the initial state radially surrounds an axial cannula portion of the first cannula (9) having the fluid outlet (8) which during the activation process pierces the first septum (11); wherein in the initial state, the second sealing envelope (23) is axially closed towards a side which faces the second coupling device (6) during the coupling process; preferably, in the initial state, the second sealing envelope (23) lies tightly against the inside of the second cannula (17).

12. The sterile connector as claimed in any of the preceding claims, characterized in that the activation process causes an axial cannula portion of the first cannula (9) to pierce the second sealing envelope (23).

13. The sterile connector as claimed in any of the preceding claims, characterized in that an actuation element (24) is provided on the cannula holder (19) for manual actuation of the cannula holder (19), by means of which the cannula holder (19) together with the second cannula (17) and the second coupling device (6) can be moved generally linearly, in particular also rotationally, relative to the first coupling device (5) during the activation process; preferably, the actuation element (24) extends radially outward from the cannula holder (19) through a guide opening (25) in a wall portion of the first coupling device (5).

14. The sterile connector as claimed in any of the preceding claims, characterized in that the actuation element (24) is axially, in particular also rotationally, movable in the guide opening (25) during the activation process; preferably, the actuation element (24) is movable in the guide opening (25) initially in the circumferential direction and then axially during the activation process; further preferably, the activation process causes the actuation element (24) to latch in the guide opening (25).

15. The sterile connector as claimed in any of the preceding claims, characterized in that the fluid passage (10) of the second coupling device (6) opens into a fluid chamber (3′) of the second coupling device (6) which has a fluid outlet (15) fluidically connected to the fluid passage (10) or which is otherwise closed.

16. The sterile connector as claimed in claim 3, characterized in that the activation process comprises a linear movement, in particular exclusively a linear movement, of the first coupling device (5) relative to the second coupling device (6).

17. The sterile connector as claimed in claim 16, characterized in that the region of the second coupling device (6) which is hermetically sealed in the initial state is delimited at least partially, preferably completely, by the first septum (11) and by a first sealing envelope (18), in particular in the form of a bellows.

18. The sterile connector as claimed in claim 16 or 17, characterized in that the second coupling device (6) has a second cannula (17) which, during the coupling process and in particular during the activation process, is arranged coaxially to the first cannula (9) and into which the first cannula (9) can be moved; preferably, the coupling process causes an axial cannula portion of the second cannula (17) to pierce the first sealing envelope (18); further preferably, the first cannula (9) and the first sealing envelope (18) do not touch one another before, during and/or after the coupling process.

19. The sterile connector as claimed in any of claims 16 to 18, characterized in that the region of the first coupling device (5) which is hermetically sealed in the initial state of the sterile connector (1) is at least partially delimited against the environment by second septum (12) and/or a second sealing envelope (23), in particular in the form of a hose closed at one end, of the first coupling device (5).

20. The sterile connector as claimed in any of claims 16 to 19, characterized in that the coupling process causes an axial cannula portion of the second cannula (17) to pierce the second septum (12) and thereby provide access for the first cannula (9) to the first septum (11); preferably, the first cannula (9) and the second septum (12) do not touch one another before, during or after the coupling process.

21. The sterile connector as claimed in any of claims 16 to 20, characterized in that the first sealing envelope (18) in initial state radially surrounds the axial cannula portion of the second cannula (17) which pierces the second septum (12) during the coupling process, wherein the first sealing envelope (18) in initial state is axially closed towards a side which faces the first coupling device (5) during the coupling process.

22. The sterile connector as claimed in any of claims 16 to 21, characterized in that the second sealing envelope (23) in the initial state radially surrounds an axial cannula portion of the first cannula (9) having the fluid outlet (8), which during the activation process pierces the first septum (11), wherein in the initial state, the second sealing envelope (23) is axially closed towards the side facing the second coupling device (6) during the coupling process.

23. The sterile connector as claimed in any of claims 16 to 22, characterized in that the coupling process causes an axial cannula portion of the first cannula (9) to pierce the second sealing envelope (23).

24. The sterile connector as claimed in any of claims 16 to 23, characterized in that the coupling process causes the end of the first cannula (9) forming the fluid outlet (8) to protrude axially into the region of the second coupling device (6) which is hermetically sealed in the initial state.

25. The sterile connector as claimed in any of claims 16 to 24, characterized in that a mechanism (31) is provided for indication, in particular haptic indication, of at least one defined axial position of the first coupling device (5) relative to the second coupling device (6); preferably, a defined axial position corresponds to the starting position in coupled state, and/or a defined axial position corresponds to the operating position in coupled state; further preferably, the first coupling device (5) can be brought from the initial state into the respective defined axial position relative to the second coupling device (6).

26. A packing arrangement with a packing (29, 30) and with, packed in sterile fashion therein, at least one, preferably precisely one, first or second coupling device (5, 6) of a sterile connector (1) as claimed in any of claims 1 to 25, or with, packed in sterile fashion therein, a sterile connector (1) as claimed in any of claims 1 to 25.

27. The packing arrangement as claimed in claim 26, characterized in that the packing (29, 30) also contains, packed in sterile fashion, an in particular filled liquid container (2) which is fluidically connected to the first coupling device (5) of the sterile connector (1), preferably via a hose (26) or tube; or, packed in sterile fashion, a bioprocess-technical system (4) which is fluidically connected to the second coupling device (6) of the sterile connector (1), preferably via a hose (26) or a tube.

28. A use of a sterile connector (1), packed in sterile fashion, as claimed in any of claims 1 to 25, a first or second coupling device (6), packed in sterile fashion, of a sterile connector (1) as claimed in any of claims 1 to 25, and/or a packing arrangement (27, 28) as claimed in claim 26 or 27, for the sterile transfer of the liquid, in particular biological medium from a liquid container (2) into a fluid chamber (3, 3′), preferably of a bioprocess-technical system (4), in particular a bioreactor, or into a fluid chamber (3′) of the second coupling device (6).

29. The use as claimed in claim 28, characterized in that at least one coupling device (5, 6), in particular both coupling devices (5, 6), preferably also the liquid container (2) and/or the bioprocess-technical system (4) and/or the hose(s) (26) or tube(s), in particular as single-use components, are made at least partially, preferably at least mainly, of a plastic material.
